(19) United States
(12) Patent Application Publication
(10) Pub. No.: US 2021/0341336 A1
(43) Pub. Date: Nov. 4, 2021

(54) DEVICE FOR DETECTING CONCENTRATION OF HEAVY METALS

(71) Applicant: Institute of Geochemistry, Chinese Academy of Sciences, Guiyang (CN)

(72) Inventors: Xiaoyong BAI, Guiyang (CN); Shijie WANG, Guiyang (CN); Shiqi TIAN, Guiyang (CN); Qian LU, Guiyang (CN); Jinfeng WANG, Guiyang (CN); Qin LI, Guiyang (CN); Luhua WU, Guiyang (CN); Yujie YANG, Guiyang (CN); Chaojun LI, Guiyang (CN); Zeyin HU, Guiyang (CN); Fei CHEN, Guiyang (CN); Yuanhong DENG, Guiyang (CN)

(21) Appl. No.: 16/862,834

(22) Filed: Apr. 30, 2020

(51) Int. Cl. G01J 3/28; G01J 3/18; G01N 21/31

(52) U.S. Cl. CPC G01J 3/2823; G01N 21/31; G01J 3/18

(57) ABSTRACT

The present invention relates to a device for detecting concentration of heavy metals. The device for detecting concentration of heavy metals acquires spectrum information of a carrier to be tested by utilizing a spectrum information acquiring system, a receiving system and a processing system. The device further processes the spectrum information to obtain the type of the carrier to be tested and the corresponding concentration of heavy metals. Therefore, the efficiency of acquiring the whole concentration of the heavy metals is remarkably improved. Furthermore, based on the specific structure of the device for detecting concentration of heavy metals provided by the present invention, the device for detecting concentration of heavy metals has the characteristics of simple structure and low costs.

### TECHNICAL FIELD

The present invention relates to the field of heavy metal detection technologies, and in particular to a device for detecting concentration of heavy metals.

### BACKGROUND

Heavy metals may be harmful to the human health finally in various manners if they are excessively accumulated in carriers such as soil, water, plant and the like. Thus, it is very important to monitor the concentration of heavy metals in different carriers.

The traditional method for acquiring information of the concentration of heavy metals mainly utilizes field sampling, laboratory physical and chemical property analysis and the like. Such method does not only consume a large amount of economical costs, but also consumes time and efforts. Moreover, it is easy to cause irreversible damage to the ecological environment.

Recently, hyperspectral imaging technology is widely applied to the field of monitoring the concentration of heavy metals in the soil due to its characteristics of quickness, high efficiency, economical efficiency, nondestructive property and the like, but there are still some limitations. Firstly, an indoor spectrometer still needs to collect a few samples, determine spectrum information of the samples by the laboratory instruments, models to analyze, and then obtains the concentration of the heavy metals. Furthermore, the indoor environment and the outdoor environment are greatly different so that the actual heavy metal pollution is hard to be reflected. Secondly, the airborne or spaceborne hyperspectral instrument has a partial coverage area, and its collected spectrum information is too redundant and needs to be preprocessed, so the obtained information is lagging. Finally, the two spectrometers have low portability, complex structure, high measurement conditions and the like, so they are hard to be used by unprofessional persons and have low generalization performance.

In conclusion, it is a technical problem urgently to be solved in the field to provide a device for detecting concentration of heavy metals, which can instantly acquire information of heavy metal concentration and has the characteristic of a simple structure.

### SUMMARY

An objective of the present invention is to provide a device for detecting concentration of heavy metals, which can improve efficiency of acquiring the concentration of heavy metals while having the characteristics of simple structure, low costs and the like.

To achieve the above objective, the present invention provides the following solution:

A device for detecting concentration of heavy metals includes a spectrum information acquiring system to acquire spectrum information of heavy metals in a carrier to be tested. A receiving system is connected with the spectrum information acquiring system and preprocesses the spectrum information and determines the type of the carrier to be tested. A processing system in communication with the receiving system determines the concentration of the heavy metals in the carrier to be tested according to the preprocessed spectrum information.

Optionally, the spectrum information acquiring system includes a probe to detect optical waves of the heavy metals in the carrier to be tested, a grating spectrometer connected with the probe and operable to separate the optical waves by diffraction to obtain a spectral band. A detector acquires spectrum information in the spectral band.

Optionally, the receiving system includes a spectrum optimizing device for optimizing the spectral band and extracting the spectrum information in the optimized spectral band. The optimization includes bad line repair, fringe removal and mixed pixel separation. A preprocessing device, connected with the spectrum optimizing device preprocesses the extracted spectrum information. The preprocessing includes abnormal value elimination, smoothing process and noise reduction.

Optionally, the processing system is a computer.

Optionally, the device for detecting concentration of heavy metals further includes a visual system connected with the processing system to display the type of the carrier to be tested and the concentration of the heavy metals, which are transmitted by the processing system.

Optionally, the visual system includes a transmitter for wirelessly transmitting the type of the carrier to be tested and the concentration of the heavy metals, a client wirelessly communicating with the transmitter and which receives and displays the type of the carrier to be tested and the concentration of the heavy metals.

Optionally, the transmitter is a Bluetooth module.

Optionally, the client is a mobile phone, a tablet computer or a laptop.

According to specific embodiments provided by the present invention, the present invention discloses the following technical effects: the device for detecting concentration of heavy metals provided by the present invention acquires the spectrum information of the carrier to be tested by utilizing the spectrum information acquiring system, the receiving system and the processing system. The device for detecting concentration of heavy metals further processes the spectrum information to obtain the type of the carrier to be tested and the corresponding concentration of the heavy metals. Therefore, the efficiency of acquiring the whole concentration of the heavy metals is remarkably improved. Furthermore, based on the specific structure of the device for detecting concentration of heavy metals provided by the present invention, the device for detecting concentration of heavy metals has the characteristics of simple structure and low costs.

### BRIEF DESCRIPTION OF THE DRAWINGS

To describe the technical solutions in the embodiments of the present invention or in the prior art more clearly, the following briefly introduces the accompanying drawings required for describing the embodiments. Apparently, the accompanying drawings in the following description show merely some embodiments of the present invention, and a person of ordinary skill in the art may still derive other accompanying drawings from these accompanying drawings without creative efforts.

FIG. 1 is a schematic structural diagram of a device for detecting concentration of heavy metals provided in embodiments of the present invention.

FIG. 2 is a schematic structural diagram of a spectrum information acquiring system provided in embodiments of the present invention.

FIG. 3 is a schematic structural diagram of a receiving system provided in embodiments of the present invention.

FIG. 4 is a workflow diagram of a device for detecting concentration of heavy metals provided in embodiments of the present invention.

### REFERENCE SIGNS

1-Charging port, 2-power supply, 3-spectrum information acquiring system, 4-receiving system, 5-processing system, 6-visual system, 31-probe, 32-grating spectrometer, 41-spectrum optimizing device, 42-preprocessing device, 51-calculating device, 61-transmitter, and 62-client.

### DESCRIPTION OF THE EMBODIMENTS

The following clearly and completely describes the technical solutions in the embodiments of the present invention with reference to accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

An objective of the present invention is to provide a device for detecting concentration of heavy metals, which can improve efficiency of acquiring the concentration of heavy metals while having the characteristics of simple structure, low costs and the like.

To make the foregoing objective, features, and advantages of the present invention more apparent and more comprehensible, the present invention is further described in detail below with reference to the accompanying drawings and specific embodiments.

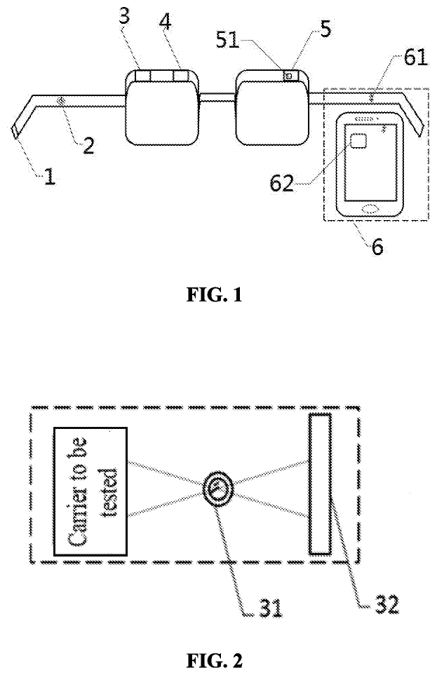

FIG. 1 is a schematic structural diagram of a device for detecting concentration of heavy metals provided in embodiments of the present invention. As shown in FIG. 1, the device for detecting concentration of heavy metals includes a spectrum information acquiring system 3, a receiving system 4 and a processing system 5.

The spectrum information acquiring system 3 is used for acquiring spectrum information of heavy metals in a carrier to be tested by utilizing hyperspectral imaging technology.

The receiving system 4 is connected or in communication with the spectrum information acquiring system 3 and is configured to preprocess the spectrum information and determine the type of the carrier to be tested.

The processing system 5 is connected or in communication with the receiving system 4 and is configured to determine the concentration of the heavy metals in the carrier to be tested according to the preprocessed spectrum information.

As shown in FIG. 2, the spectrum information acquiring system 3 includes a probe 31, a grating spectrometer 32 and a detector.

Optical waves of the carrier to be tested pass through the probe 31 and then are separated in diffraction by the grating spectrometer 32 such that a compound light with different wavelengths is dispersed into independent monochromatic lights. The monochromatic lights are sequenced in a descending order to form a spectral band. Then the spectrum information in the spectral band is acquired by the detector.

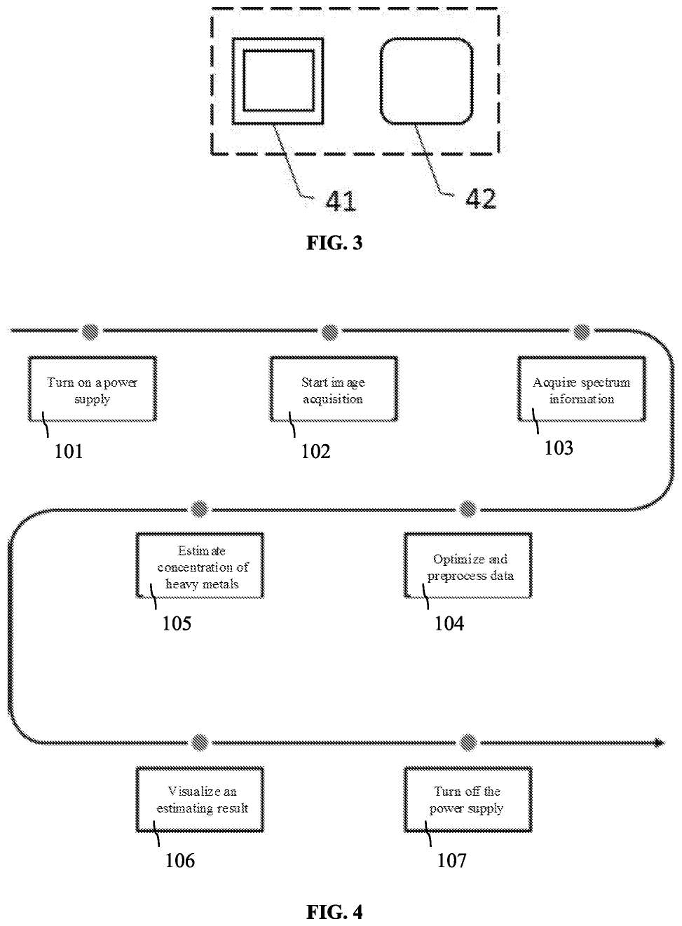

As shown in FIG. 3, the receiving system 4 includes a spectrum optimizing device 41 and a preprocessing device 42.

The spectrum optimizing device 41 conducts optimization including bad line repair, fringe removal and mixed pixel separation on the acquired spectral band, and extracts effective spectrum information in the optimized spectral band.

The preprocessing device 42 is connected or in communication with the spectrum optimizing device 41 and stores preprocessed spectrum data after conducting preprocessing including abnormal value elimination, smoothing process and noise reduction on the extracted spectrum information in the spectral band. Additionally, the type of the carrier to be tested should be identified to soil, water, vegetation and the like after the spectrum information is preprocessed.

The whole preprocessing includes: First, based on the optimization on the spectral band, combining standard score (Z-score) with principal component analysis (PCA) to further screen a spectrum outlier, and eliminating the spectrum outlier as an abnormal value to ensure accuracy of a sample and an estimating result; Second, combining median filter with Savitzky-Golay smoothing filter to conduct noise reduction, convolution and smoothing on outlier-eliminated spectrum data; Third, on the basis of this, resampling the spectrum data at an interval of 10 nm, and utilizing the data as conversion basic data; and Fourth, conducting different forms of conversion on the basic data, wherein the different forms of conversion can include reflectance first-order differentiation, reflectance second-order differentiation, absorbance conversion, absorbance first-order differentiation, absorbance second-order differentiation, multiplicative scatter correction (MSC), standard normal variate (SNV) and the like, and the basic data is the reflectance of an original spectrum. To a certain extent, spectrum translation caused by moisture absorption can be eliminated, the spectrum information is amplified, colinearity of the spectrum data is improved, overfitting is prevented, and stability of a model is improved. Generally, the whole preprocessing may be autonomously completed by utilizing preset program codes.

The processing system 5 includes a calculating device 51 in which a heavy metal concentration calculation model is set. The processing system automatically operates a pre-programmed algorithm code input program to complete accurate estimation on the concentration of the heavy metals.

The heavy metal concentration calculation model is mainly divided into a linear model and a nonlinear model. The linear model includes, but is not limited to, a multiple stepwise regression model, a least squares model, a principal component regression model and the like. The nonlinear model includes, but is not limited to, a neural network model, a random forest model, a support vector machine model, an extreme learning machine model and the like.

The processing system 5 automatically selects an optimal model according to target heavy metals to be detected. The optimal model utilizes the spectrum data screened and preprocessed by the receiving system 4 as input data and the concentration of the heavy metals as output data, and such process is mainly autonomously completed based on the preset program codes.

Furthermore, the processing system 5 may be replaced with a computer to complete autonomous operation of the program codes.

The device for detecting concentration of heavy metals further includes a visual system 6 connected or in communication with the processing system 5 and used for displaying the type of the carrier to be tested and the concentration of the heavy metals, which are transmitted by the processing system 5.

The visual system 6 includes a transmitter 61 and a client 62.

The transmitter 61 is used for wirelessly transmitting the type of the carrier to be tested and the concentration of the heavy metals.

The client 62 wirelessly communicates with the transmitter 61 and is configured to receive and display the type of the carrier to be tested and the concentration of the heavy metals.

In one embodiment, the transmitter 61 is a Bluetooth module and the client 62 is a mobile phone, a tablet computer or a laptop.

The client can preset data including a background value, recommended daily allowance and the like to be compared with the result of the processing system 5 and provides a rational suggestion according to a comparison result.

The background value can input soil element background value data issued by a certain country or region and can also be replaced according to the newest research result. The recommended daily allowance can input data corresponding to the rational supply standard planned according to the research development and the domestic nutritional status.

An example of the comparison with the result of the processing system 5 is as follows: (1) if the processing system 5 calculates an average of the concentration of Pb in multiple soils of a certain region to be 5 mg/kg, but a preset Pb background value is 35 mg/kg, the region does not have a Pb pollution situation; (2) if the processing system 5 calculates the concentration of Zn in the vegetable is 3 mg, but the preset recommended daily allowance of Zn is 15 mg, five pieces of such vegetable can be eaten daily.

Therefore, the client can provide rational pollution prevention suggestions or scientific dietary pattern suggestions according to the above results.

Furthermore, to help the whole device for detecting concentration of heavy metals to be carried and applied, the device for detecting concentration of heavy metals can be further equipped with a charging port 1 and a power supply 2. The charging port 1 is used for charging the power for the whole device for detecting concentration of heavy metals. The power supply 2 powers the whole device.

As shown in FIG. 4, according to the whole workflow of the device for detecting concentration of heavy metals provided by the present invention, when the concentration of the heavy metals needs to be detected, the power supply 2 is firstly turned on (101). Secondly, the spectrum information acquiring system 3 starts working (102). The probe 31 acquires carrier information, the grating spectrometer 32 separates the light by diffraction to form the spectral band (103), and the spectrum information is outputted to the receiving system 4. Thirdly, the receiving system 4 optimizes and preprocesses the spectrum information (105) acquired by the spectrum information acquiring system 3, classifies the carrier to be tested, utilizes an algorithm to conduct abnormal value elimination, smoothing and noise reduction on the spectrum, and finally feeds back the result to the processing system 5. Fourthly, the processing system 5 calculates the concentration of the heavy metals (105) by utilizing the preset calculation module, and transmits the calculation result to the client through the transmitter 61 in order to reflect specific concentration information of the heavy metals and display the same on the client 62 (106). The processing system 5 can compare the calculating result with related indexes and provides the rational suggestion. Finally, after the detection is completed and visualization provided, the power supply 2 is turned off (107).

Based on the solution, the present invention provides the device for detecting concentration of heavy metals, which is based on the hyperspectral imaging technology. The device for detecting concentration of heavy metals combines the response relationship of the heavy metals and the spectrum to achieve instant acquisition of the concentration of the heavy metals. The device also effectively solves the problems that the traditional sampling method consumes time and efforts, has low efficiency and high costs and the like. Furthermore, the device for detecting concentration of heavy metals provided by the present invention further has the characteristics of simple operation, instant information acquisition, and high popularization and application values.

Each embodiment of the present specification is described in a progressive manner, each embodiment focuses on the difference from other embodiments, and the same and similar parts between the embodiments may refer to each other.

Several examples are used for illustration of the principles and implementation methods of the present invention. The description of the embodiments is used to help illustrate the method and its core principles of the present invention. In addition, a person of ordinary skill in the art can make various modifications in terms of specific embodiments and scope of application in accordance with the teachings of the present invention. In conclusion, the content of this specification shall not be construed as a limitation to the present invention.

What is claimed is:

1. A device for detecting concentration of heavy metals, comprising:
a spectrum information acquiring system configured to acquire spectrum information of heavy metals in a carrier to be tested;
a receiving system connected with the spectrum information acquiring system and configured to preprocess the spectrum information and determine a type of the carrier to be tested; and
a processing system connected with the receiving system and configured to determine the concentration of the heavy metals in the carrier to be tested according to the preprocessed spectrum information.

2. The device for detecting concentration of heavy metals according to claim 1, wherein the spectrum information acquiring system comprises:
a probe for detecting optical waves of the heavy metals in the carrier to be tested;
a grating spectrometer connected with the probe and configured to separate the detected optical waves by diffraction to obtain a spectral band; and
a detector connected with the grating spectrometer and configured to acquire spectrum information in the spectral band.

3. The device for detecting concentration of heavy metals according to claim 2, wherein the receiving system comprises:
a spectrum optimizing device configured to optimize the spectral band and extract the spectrum information in the optimized spectral band, wherein the optimization comprises bad line repair, fringe removal and mixed pixel separation; and
a preprocessing device connected with the spectrum optimizing device and configured to preprocess the extracted spectrum information, wherein the preprocessing comprises abnormal value elimination, smoothing process and noise reduction.

4. The device for detecting concentration of heavy metals according to claim 1, wherein the processing system comprises a computer.

5. The device for detecting concentration of heavy metals according to claim 1, further comprising a visual system connected with the processing system and configured to display the type of the carrier to be tested and the concentration of the heavy metals, which are transmitted by the processing system.

6. The device for detecting concentration of heavy metals according to claim 5, wherein the visual system comprises:
a transmitter configured to wirelessly transmit the type of the carrier to be tested and the concentration of the heavy metals; and
a client in wireless communication with the transmitter and configured to receive and display the type of the carrier to be tested and the concentration of the heavy metals.

7. The device for detecting concentration of heavy metals according to claim 6, wherein the transmitter comprises a Bluetooth module.

8. The device for detecting concentration of heavy metals according to claim 6, wherein the client comprises one of a mobile phone, a tablet computer or a laptop.
